(19) United States
(12) Patent Application Publication
(10) Pub. No.: US 2012/0328714 A1
(43) Pub. Date: Dec. 27, 2012

(54) EARLY DETECTION AND TREATMENT OF LUNG CANCER

(76) Inventors: Ping Yang, Rochester, MN (US); Liang Wang, Rochester, MN (US)

(21) Appl. No.: 13/475,333

(22) Filed: May 18, 2012

Related U.S. Application Data
(60) Provisional application No. 61/487,544, filed on May 18, 2011.

(51) Int. Cl. C12Q 1/68; A61P 35/00; A61N 5/10; A61K 33/24; A61K 31/282; A61K 31/437; A61K 31/7048; A61K 31/4545

(52) U.S. Cl. 424/649; 514/27; 435/6.11; 514/283; 514/492; 600/1

(57) ABSTRACT

This document provides methods and materials involved in the early detection of lung cancer (e.g., small cell lung cancer). For example, this document provides methods and materials for assessing nucleic acid obtained from a blood sample of a human for a CpG methylation site profile that, at least in part, indicates that the human has lung cancer (e.g., small cell lung cancer).

### CROSS-REFERENCE TO RELATED APPLICATIONS

This application claims the benefit of U.S. Provisional Application Ser. No. 61/487,544, filed May 18, 2011. The disclosure of the prior application is considered part of (and is incorporated by reference in) the disclosure of this application.

### STATEMENT AS TO FEDERALLY SPONSORED RESEARCH

This invention was made with government support under grants CA080127; CA084354; and CA077118 awarded by the National Institutes of Health. The government has certain rights in the invention.

### BACKGROUND

1. Technical Field

This document relates to methods and materials involved in the early detection and treatment of lung cancer (e.g., small cell lung cancer). For example, this document provides methods and materials for assessing nucleic acid obtained from a blood sample of a human for a CpG methylation site profile that, at least in part, indicates that the human has lung cancer (e.g., small cell lung cancer).

2. Background Information

Small-cell lung cancer (SCLC) constitutes approximately 13 percent of all newly diagnosed lung cancers. In comparison to the more common non-small cell lung cancer (NSCLC), SCLC has more rapid doubling time, higher growth fraction, earlier development of widespread metastases, and more dramatic initial response to chemotherapy and radiation. Despite high initial responses to therapy, most patients die from recurrent disease. Untreated SCLC has the most aggressive clinical course of any lung tumor, with a median survival of only 2 to 4 months after diagnosis. Cigarette smoking is the strongest risk factor for the development of SCLC. Virtually all patients with SCLC are current or past smokers, and its risk appears to be related to the duration and intensity of the smoking.

### SUMMARY

This document provides methods and materials involved in the early detection and treatment of lung cancer (e.g., small cell lung cancer). For example, this document provides methods and materials for assessing nucleic acid obtained from a blood sample of a human for a CpG methylation site profile that, at least in part, indicates that the human has lung cancer (e.g., small cell lung cancer) as well as provides methods and materials for treating lung cancer patient at an early point in the patient's development of lung cancer. In some cases, a lung cancer patient can be treated for lung cancer following the early detection of lung cancer by assessing nucleic acid obtained from a blood sample for a CpG methylation site profile that, at least in part, indicates that the human has lung cancer (e.g., small cell lung cancer).

As described herein, nucleic acid from blood cells of humans with lung cancer (e.g., small cell lung cancer) can contain different levels of the methylation CpG sites listed in Table 1 (or Table 4 with the exception of the CAV1 gene) when compared to the level of methylation of those CpG sites in nucleic acid from blood cells of humans without lung cancer. In particular, the methylation change in at least three methylation CpG sites listed in Table 1 (e.g., IL10_P85_F, PECAM1_E32_R, S100A2_E36_R, MMP9_P189_F, ERCC1_P440_R, EMR3_E61_F, SLC22A18_P216_R, TRIP6_P1090_F, and CSF3R_P472_F CpG methylation sites) can indicate that a human has lung cancer (e.g., small cell lung cancer).

The methods and materials provided herein can allow clinicians to detect humans with lung cancer (e.g., small cell lung cancer) at an early stage without the need to obtain invasive tissue biopsies (e.g., lung tissue biopsies). Such an early detection can allow patients to be treated sooner with the hopes that a successful treatment outcome will be achieved.

In general, one aspect of this document provides a method for identifying a human as having small cell lung cancer. The method comprises, or consists essentially of, (a) performing a bisulfite conversion using nucleic acid obtained from a blood sample of a human to detect at least three methylation CpG sites that have an altered methylation status indicative of small cell lung cancer, wherein the at least three methylation CpG sites are selected from the group consisting of the CpG methylation sites listed in Table 1, and (b) classifying the human as having small cell lung cancer based at least in part on the detection of the at least three methylation CpG sites that have an altered methylation status indicative of small cell lung cancer. The blood sample can be a blood sample obtained from a human not subjected to a prior lung tissue biopsy. The method can comprise performing the bisulfite conversion using the nucleic acid to detect at least four methylation CpG sites selected from the group that have an altered methylation status indicative of small cell lung cancer. The at least four methylation CpG sites can be selected from the group consisting of IL10_P85_F, PECAM1_E32_R, S100A2_E36_R, MMP9_P189_F, ERCC1_P440_R, EMR3_E61_F, SLC22A18_P216_R, TRIP6_P1090_F, and CSF3R_P472_F CpG methylation sites. The method can comprise performing the bisulfite conversion using the nucleic acid to detect at least five methylation CpG sites selected from the group that have an altered methylation status indicative of small cell lung cancer. The at least five methylation CpG sites can be selected from the group consisting of IL10_P85_F, PECAM1_E32_R, S100A2_E36_R, MMP9_P189_F, ERCC1_P440_R, EMR3_E61_F, SLC22A18_P216_R, TRIP6_P1090_F, and CSF3R_P472_F CpG methylation sites. The method can comprise performing the bisulfite conversion using the nucleic acid to detect IL10_P85_F, PECAM1_E32_R, S100A2_E36_R, MMP9_P189_F, ERCC1_P440_R, EMR3_E61_F, SLC22A18P216_R, TRIP6_P1090_F, and CSF3R_P472_F CpG methylation sites that have an altered methylation status indicative of small cell lung cancer.

In another aspect, this document features a method for treating small cell lung cancer. The method comprises, or consists essentially of, (a) detecting the presence of at least three methylation CpG sites that have an altered methylation status indicative of small cell lung cancer in nucleic acid obtained from a blood sample of a human, wherein the at least three methylation CpG sites are selected from the group consisting of the CpG methylation sites listed in Table 1, and (b) administering a cancer radiation treatment, a cancer chemotherapeutic agent, or a combination thereof to the human. The blood sample can be a blood sample obtained from a human not subjected to a prior lung tissue biopsy. The method can comprise detecting the presence of at least four methylation CpG sites selected form the group that have an altered methylation status indicative of small cell lung cancer in the nucleic acid. The at least four methylation CpG sites can be selected from the group consisting of IL10_P85_F, PECAM1_E32_R, S100A2_E36_R, MMP9_P189_F, ERCC1_P440_R, EMR3_E61_F, SLC22A18_P216_R, TRIP6_P1090_F, and CSF3R_P472_F CpG methylation sites. The method can comprise detecting the presence of at least five methylation CpG sites selected from the group that have an altered methylation status indicative of small cell lung cancer in the nucleic acid. The at least five methylation CpG sites can be selected from the group consisting of IL10_P85_F, PECAM1_E32_R, S100A2_E36_R, MMP9_P189_F, ERCC1_P440_R, EMR3_E61_F, SLC22A18_P216_R, TRIP6_P1090_F, and CSF3R_P472_F CpG methylation sites. The method can comprise detecting the presence of IL10_P85_F, PECAM1_E32_R, S100A2_E36_R, MMP9_P189_F, ERCC1_P440_R, EMR3_E61_F, SLC22A18_P216_R, TRIP6_P1090_F, and CSF3R_P472_F CpG methylation sites that have an altered methylation status indicative of small cell lung cancer in the nucleic acid. The method can comprise administering the cancer radiation treatment. The cancer radiation treatment can comprise stereotactic body radiotherapy. The method can comprise administering the cancer chemotherapeutic agent. The cancer chemotherapeutic agent can be etoposide, irinotecan, cisplatin, carboplatin, vincristine sulfate, or a combination thereof.

In another aspect, this document features a method for identifying a human as having small cell lung cancer. The method comprises, or consists essentially of, (a) determining whether or not nucleic acid obtained from a blood sample of a human comprises at least three methylation CpG sites that have an altered methylation status indicative of small cell lung cancer, wherein the at least three methylation CpG sites are selected from the group consisting of the CpG methylation sites listed in Table 1, and (b) classifying the human as having small cell lung cancer if the nucleic acid comprises the at least three methylation CpG sites that have an altered methylation status indicative of small cell lung cancer, and classifying the human as not having small cell lung cancer if the nucleic acid does not comprise the at least three methylation CpG sites that have an altered methylation status indicative of small cell lung cancer. The blood sample can be a blood sample obtained from a human not subjected to a prior lung tissue biopsy. The method can comprise determining whether or not nucleic acid obtained from the blood sample comprises at least four methylation CpG sites selected from the group that have an altered methylation status indicative of small cell lung cancer. The at least four methylation CpG sites can be selected from the group consisting of IL10_P85_F, PECAM1_E32_R, S100A2_E36_R, MMP9_P189_F, ERCC1_P440_R, EMR3_E61_F, SLC22A18P216_R, TRIP6_P1090_F, and CSF3R_P472_F CpG methylation sites. The method can comprise determining whether or not nucleic acid obtained from the blood sample comprises at least five methylation CpG sites selected from the group that have an altered methylation status indicative of small cell lung cancer. The at least five methylation CpG sites can be selected from the group consisting of IL10_P85_F, PECAM1_E32_R, S100A2_E36_R, MMP9_P189_F, ERCC1_P440_R, EMR3_E61_F, SLC22A18P216_R, TRIP6_P1090_F, and CSF3R_P472_F CpG methylation sites. The method can comprise determining whether or not nucleic acid obtained from the blood sample comprises IL10_P85_F, PECAM1_E32_R, S100A2_E36_R, MMP9_P189_F, ERCC1_P440_R, EMR3_E61_F, SLC22A18_P216_R, TRIP6_P1090_F, and CSF3R_P472_F CpG methylation sites that have an altered methylation status indicative of small cell lung cancer.

In another aspect, this document features a method for identifying a human as having small cell lung cancer. The method comprises, or consists essentially of, (a) detecting the presence of at least three methylation CpG sites that have an altered methylation status indicative of small cell lung cancer in nucleic acid obtained from a blood sample of a human, wherein the at least three methylation CpG sites are selected from the group consisting of the CpG methylation sites listed in Table 1, and (b) classifying the human as having small cell lung cancer based at least in part on the presence of the at least three methylation CpG sites that have an altered methylation status indicative of small cell lung cancer. The blood sample can be a blood sample obtained from a human not subjected to a prior lung tissue biopsy. The method can comprise detecting the presence of at least four methylation CpG sites selected form the group that have an altered methylation status indicative of small cell lung cancer in the nucleic acid. The at least four methylation CpG sites can be selected from the group consisting of IL10_P85_F, PECAM1_E32_R, S100A2_E36_R, MMP9_P189_F, ERCC1_P440_R, EMR3_E61_F, SLC22A18_P216_R, TRIP6_P1090_F, and CSF3R_P472_F CpG methylation sites. The method can comprise detecting the presence of at least five methylation CpG sites selected from the group that have an altered methylation status indicative of small cell lung cancer in the nucleic acid. The at least five methylation CpG sites can be selected from the group consisting of IL10_P85_F, PECAM1_E32_R, S100A2_E36_R, MMP9_P189_F, ERCC1_P440_R, EMR3_E61_F, SLC22A18P216_R, TRIP6_P1090_F, and CSF3R_P472_F CpG methylation sites. The method can comprise detecting the presence of IL10_P85_F, PECAM1_E32_R, S100A2_E36_R, MMP9_P189_F, ERCC1_P440_R, EMR3_E61_F, SLC22A18P216_R, TRIP6_P1090_F, and CSF3R_P472_F CpG methylation sites that have an altered methylation status indicative of small cell lung cancer in the nucleic acid.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is contains graphs of a methylation analysis of IL10_P85_F CpG in three representative samples by pyrosequencing technology. The first arrow at T position indicates signal peak of unmethylated C. The second arrow at C position represents signal peak of methylated C. The CpG methylation level is the percentage of methylated C among the sum of methylated C and unmethylated C. The methylation levels for the samples 1, 2, and 3 are 2%, 21%, and 82%, respectively.

FIG. 2 is a graph plotting an ROC curve in the validation set of 138 cases and 138 controls using nine CpGs selected from the set. The curve illustrates the capacity of the methylation levels of the CpGs to discriminate between SCLC cases and controls. The area under the ROC curve (the c-statistic), represents the proportion of SCLC case-control pairs where the case is predicted by the model based on the nine CpGs to have greater odds of being a SCLC case.

### DETAILED DESCRIPTION

This document provides methods and materials involved in the early detection and treatment of lung cancer (e.g., small cell lung cancer). For example, this document provides methods and materials for assessing nucleic acid obtained from a blood sample of a human for a CpG methylation site profile that, at least in part, indicates that the human has lung cancer (e.g., small cell lung cancer) as well as provides methods and materials for treating lung cancer patient at an early point in the patient's development of lung cancer. In some cases, a lung cancer patient can be treated for lung cancer following the early detection of lung cancer by assessing nucleic acid obtained from a blood sample for a CpG methylation site profile that, at least in part, indicates that the human has lung cancer (e.g., small cell lung cancer).

As described herein, nucleic acid from blood samples of humans with lung cancer (e.g., small cell lung cancer) can contain different levels of methylation at particular CpG sites (e.g., the methylation CpG sites listed in Table 1 or the methylation CpG sites listed in Table 4 with the exception of the CAV1 gene) when compared to nucleic acid from blood samples of humans without lung cancer. The methylation level change in these methylated CpG sites can be used to identify humans with lung cancer (e.g., small cell lung cancer). For example, methylation level changes in at least three (e.g., at least four, at least five, at least six, at least seven, at least eight, at least nine, or at least ten) methylation CpG sites listed in Table 1 (or Table 4 with the exception of the CAV1 gene) can indicate that a human has lung cancer (e.g., small cell lung cancer). In some cases, methylation level changes in the methylation CpG sites listed in Table 1 (or Table 4 with the exception of the CAV1 gene) can indicate that a human has lung cancer (e.g., small cell lung cancer). In some cases, methylation level changes in any three, four, five, six, seven, or eight of the following CpG methylation sites can indicate that a human has lung cancer (e.g., small cell lung cancer): IL10_P85_F, PECAM1_E32_R, S100A2_E36_R, MMP9_P189_F, ERCC1_P440_R, EMR3_E61_F, SLC22A18_P216_R, TRIP6_P1090_F, and CSF3R_P472_F CpG methylation sites. In some cases, methylation level changes in each the following nine CpG methylation sites can indicate that a human has lung cancer (e.g., small cell lung cancer): IL10_P85_F, PECAM1_E32_R, S100A2_E36_R, MMP9_P189_F, ERCC1_P440_R, EMR3_E61_F, SLC22A18P216_R, TRIP6_P1090_F, and CSF3R_P472_F CpG methylation sites.

TABLE 1
Selected CpG sites.

| | | | | | Methylation | |
|---|---|---|---|---|---|---|
| | | GenBank ® | | | change in | SEQ |
| | Illumina | Accession | GenBank ® | Sequence of | cancer | ID |
| Symbol | CpG ID | No. | GI No. | CpG region | patients | NO: |
| SLC22A18 | SLC22A18_P216_R | NM_002555.3 | 34734074 | GTCAGCCTGGATCCTCTC | hypomethylation | 1 |
| | | | | ATC[CG]GCAGAACTGTC | | |
| | | | | GCCTTGCTTCTCTGAAGC | | |
| | | | | G | | |
| PADI4 | PADI4_E24_F | NM_012387.1 | 6912575 | TCCTACAGCCAGAGGGA | hypomethylation | 2 |
| | | | | CGAGCTAGCCCGA[CG]A | | |
| | | | | TGGCCCAGGGGACATTG | | |
| | | | | ATC | | |
| MMP9 | MMP9_P189_F | NM_004994.2 | 74272286 | TTGCCTGACTTGGCAGTG | hypomethylation | 3 |
| | | | | GAGACTG[CG]GGCAGTG | | |
| | | | | GAGAGAGGAGG | | |
| LTB4R | LTB4R_P163_F | NM_181657.1 | 31881791 | GGGGAAGAAAGGCCATC | hypomethylation | 4 |
| | | | | AAGGTAGATG[CG]GGTG | | |
| | | | | GGGAACAGCTTGAG | | |
| S100A2 | S100A2_E36_R | NM_005978.3 | 45269153 | CACAGTGGGAAGTGGGA | hypomethylation | 5 |
| | | | | GGTGT[CG]TGGGGACTG | | |
| | | | | GGCATCCTG | | |
| RUNX3 | RUNX3_P247_F | NM_001031680.1 | 72534651 | CGGCCTTGGCTCATTGGC | hypermethylation | 6 |
| | | | | TGGGCCG[CG]GTCACCT | | |
| | | | | GGGCCGTGATGTCACGG | | |
| | | | | CC | | |
| MPO | MPO_E302_R | NM_000250.1 | 4557758 | GGAGCAGCACCTTCAGA | hypomethylation | 7 |
| | | | | GGGCTGGGG[CG]TGGCC | | |
| | | | | AGAATGGCCAGGAGCCC | | |
| IL10 | IL10_P85_F | NM_000572.2 | 24430216 | AGCCACAATCAAGGTTT | hypomethylation | 8 |
| | | | | CC[CG]GCACAGGATTTTT | | |
| | | | | TCTGCTTAGAGCTCCT | | |
| RUNX3 | RUNX3_E27_R | NM_001031680.1 | 72534651 | CGGCAGCCAGGGTGGAG | hypermethylation | 9 |
| | | | | GAGCTC[CG]AAGCTGAC | | |
| | | | | AGAGCAGAGTGGGCC | | |
| PECAM1 | PECAM1_E32_R | NM_000442.2 | 21314616 | GCGCCTGCAGAGAGACC | hypomethylation | 10 |
| | | | | GGCTGTGG[CG]CTGGTC | | |
| | | | | AGGTAATGGCAGCCATG | | |
| | | | | G | | |
| EMR3 | EMR3_E61_F | NM_152939.1 | 23397638 | AGCAAACTGCTTCCCCTC | hypomethylation | 11 |
| | | | | TTT[CG]CCATCAGACTCA | | |
| | | | | TGGTTCTGCTTTTCGTTT | | |
| SPI1 | SPI1_E205_F | NM_003120.1 | 4507174 | GGGAAACCCTTCCATTTT | hypomethylation | 12 |
| | | | | GCA[CG]CCTGTAACATCC | | |
| | | | | AGCCGGGCTCCGA | | |
| TNFRSF1A | TNFRSF1A | NM_001065.2 | 23312372 | TCCTGGCTCTGCCACCAA | hypomethylation | 13 |
| | P678 F | | | TCATG[CG]ACATCAGGC | | |
| | | | | AACTCCTCTCCTAAGC | | |
| LMO2 | LMO2_E148_F | NM_005574.2 | 6633806 | CGGAGCCTTCACCCTTGC | hypomethylation | 14 |
| | | | | AG[CG]AGCTCTCTCACAC | | |
| | | | | CAGATGTGCTCTGCGT | | |
| IL10 | IL10_P348_F | NM_000572.2 | 24430216 | ATTCGCGTGTTCCTAGGT | hypomethylation | 15 |
| | | | | CACAGTGA[CG]TGGACA | | |
| | | | | AATTGCCCATTCCAGAAT | | |
| | | | | AC | | |
| ERCC1 | ERCC1_P440_R | NM_001983.2 | 42544170 | GAGCTTACGGTTCAGTA | hypomethylation | 16 |
| | | | | AGGGACACAGACA[CG]T | | |
| | | | | TCCCAGTGCTGACCCAG | | |
| | | | | AATGGG | | |
| CSF3R | CSF3R_P472_F | NM_172313.1 | 27437044 | CTCACTGCTCCCCTCTTC | hypomethylation | 17 |
| | | | | ATTA[CG]TATTCTGTGCA | | |
| | | | | TTGCCCATAGACCAGGC | | |
| | | | | A | | |
| JAK3 | JAK3_P1075_R | NM_000215.2 | 47157314 | GGACAGGCACAGACTGG | hypomethylation | 18 |
| | | | | AACTTGGACC[CG]AGGC | | |
| | | | | AGGACAGGGAGCTGGC | | |
| LCN2 | LCN2_P141_R | NM_005564.2 | 38455401 | AATGTCCCTCACTCTCCC | hypomethylation | 19 |
| | | | | [CG]TCCCTCTGTCTTGCC | | |
| | | | | CAATCCTGAC | | |
| CD82 | CD82_P557_R | NM_002231.3 | 67782352 | AAAGTTCCTGGGCCCAG | hypomethylation | 20 |
| | | | | GC[CG]CCTCCTGATAGA | | |
| | | | | GGCCCCGACTTAGG | | |
| PI3 | PI3_P274_R | NM_002638.2 | 31657130 | TCTACCAGTGACTTGCTG | hypomethylation | 21 |
| | | | | AATAACCTT[CG]GTGATT | | |
| | | | | CCTTTCTCTTCTTGGGTC | | |
| | | | | TCACT | | |
| TRIP6 | TRIP6_P1090_F | NM_003302.1 | 23308730 | AAGGGGACTTTGTGAAC | hypomethylation | 22 |
| | | | | AGTGGG[CG]GGGAGACG | | |
| | | | | CAGAGGCAGAGG | | |
| TIE1 | TIE1_E66_R | NM_005424.2 | 31543809 | CCAGCTCGTCCTGGCTGG | hypomethylation | 23 |
| | | | | CCTGGGT[CG]GCCTCTGG | | |
| | | | | AGTATGGTCTGGCGGGT | | |
| | | | | GCCCC | | |
| TRIP6 | TRIP6_P1274_R | NM_003302.1 | 23308730 | CTTGGGCATGGTGCCCGC | hypomethylation | 24 |
| | | | | TTGGCATAG[CG]CCCGG | | |
| | | | | CTCCGGATCTTCCTGTGC | | |
| | | | | CT | | |
| CD9 | CD9_P585_R | NM_001769.2 | 21237762 | CTGTCATCCCACCCAGAC | hypomethylation | 25 |
| | | | | TG[CG]CGCTTCTAATTCC | | |
| | | | | TCCTACCCCAC | | |
| SEPT9. | SEPT9_P374_F | NM_006640.2 | 19923366 | GGGGCCAGCCCAGGACA | hypomethylation | 26 |
| | | | | GAGGAAGG[CG]AGGCAG | | |
| | | | | GCACGCAGGAACTGG | | |
| MPL | MPL_P62_F | NM_005373.1 | 4885490 | AGGGGCAGGGACAGGGA | hypomethylation | 27 |
| | | | | CAGGA[CG]TGGGGCTGT | | |
| | | | | ATCTGACAGGA | | |
| CASP10 | CASP10_P334_F | NM_001230.3 | 47078266 | TGTGGACATAAGAAAGG | hypomethylation | 28 |
| | | | | GTTAACATGGC[CG]ACA | | |
| | | | | ACTATTTCATGAGCTTTT | | |
| | | | | TGGCTT | | |
| AIM2 | AIM2_P624_F | NM_004833.1 | 4757733 | GTCAGCAGTCAGCCAAG | hypomethylation | 29 |
| | | | | TTTT[CG]ACCATCTTGGC | | |
| | | | | TTTAACCAGTTGCGGCC | | |
| SEPT9. | SEPT9_P58_R | NM_006640.2 | 19923366 | CCGGTGGTCTGCCGGACT | hypomethylation | 30 |
| | | | | CCT[CG]GGGCCCACTTCG | | |
| | | | | GGCCCTCTCT | | |
| CSF1R | CSF1R_E26_F | NM_005211.2 | 27262658 | TTCTCCTCACTTCGTGCT | hypomethylation | 31 |
| | | | | CTCA[CG]CTTTTGGACAC | | |
| | | | | TCTGTCTGCCCTTCTCC | | |
| CSF3R | CSF3R_P8_F | NM_172313.1 | 27437044 | GCTTCTCTCCCCGAGCTC | hypomethylation | 32 |
| | | | | TGT[CG]TTAATGGCTCAG | | |
| | | | | CCTCTGACAGGCCCG | | |
| MMP14 | MMP14_P13_F | NM_004995.2 | 13027797 | AGGGAGGGACCAGAGGA | hypomethylation | 33 |
| | | | | GAGAG[CG]AGAGAGGGA | | |
| | | | | ACCAGACCCCAGTTCG | | |
| BTK | BTK_P105_F | NM_000061.1 | 4557376 | GCAGCATGCTATCTGGTT | hypomethylation | 34 |
| | | | | CCCTGCTGC[CG]TCCCTA | | |
| | | | | TTCCACCCCCTCAAC | | |
| GRB7 | GRB7_E71_R | NM_001030002.1 | 71979666 | GCCTCTGACTTCTCTGTC | hypomethylation | 35 |
| | | | | CGAAGT[CG]GGACACCC | | |
| | | | | TCCTACCACCTGTAGAG | | |
| STAT5A | STAT5A_P704_R | NM_003152.2 | 21618341 | CAGCCACCGACAGGCTG | hypomethylation | 36 |
| | | | | CATGA[CG]GTGGCAAAG | | |
| | | | | TCACTTCCCCTCTCTG | | |
| NOTCH4 | NOTCH4_E4_F | NM_004557.3 | 55770875 | CCTCGGCCTGCTGCAAGC | hypomethylation | 37 |
| | | | | CTCA[CG]TCTGAGCTGTT | | |
| | | | | TCCTGAGTCACACAATGT | | |
| | | | | C | | |
| HOXB2 | HOXB2_P99_F | NM_002145.2 | 24497527 | TCTATTAAACCCAGGACT | hypomethylation | 38 |
| | | | | CCAG[CG]AAATTACAGG | | |
| | | | | GAATTCGTGGTCACGGG | | |
| | | | | ACC | | |
| MFAP4 | MFAP4_P197_F | NM_002404.1 | 23111004 | GACCACCTGTGTCTCATT | hypomethylation | 39 |
| | | | | AGTCCTGT[CG]GGCAAA | | |
| | | | | GTACTGCAGACGTTAACT | | |
| | | | | CCCTGC | | |
| SLC5A5 | SLC5A5_E60_F | NM_000453.1 | 4507034 | GGACAGACAGCCGGCTG | hypomethylation | 40 |
| | | | | CATGGGACAG[CG]GAAC | | |
| | | | | CCAGAGTGAGAGGGG | | |
| CD34 | CD34_P339_R | NM_001025109.1 | 68342037 | ATCCTGTGCTGTGTGTGA | hypomethylation | 41 |
| | | | | GTGAAG[CG]TCAGGAGT | | |
| | | | | GAGCAGGTATACGTGAC | | |
| | | | | T | | |
| MFAP4 | MFAP4_P10_R | NM_002404.1 | 23111004 | TGCTCAGAGTGGCTGGG | hypomethylation | 42 |
| | | | | TGTCTG[CG]GCCCCAGAC | | |
| | | | | TGCAACCGCCCAGAGTT | | |
| EMR3 | EMR3_P39_R | NM_152939.1 | 23397638 | GGGATGATTGAGTTGGT | hypomethylation | 43 |
| | | | | AAACCCTAA[CG]AGGAA | | |
| | | | | ATGCCCTGAAAGTTACAT | | |
| | | | | CAC | | |

Any appropriate method can be used to obtain a blood sample that can be processed to obtain nucleic acid for the assessment of the human's CpG methylation site profile. For example, leukocyte nucleic acid can be obtained and assessed as described herein to determine whether any one or more of the methylation CpG sites listed in Table 1 (or Table 4 with the exception of the CAV 1 gene) have an altered level of methylation as compared to controls (e.g., healthy humans known to not have lung cancer). Any appropriate method can be used to assess a methylation CpG site for methylation level change (e.g., the presence or absence of a methyl group). For example, high-performance capillary electrophoresis, methylation-sensitive arbitrarily primed PCR, and a bisulfite conversion method can be used to determine the methylation state of methylation CpG sites. In some cases, methylation assays available commercially (e.g., from Illumina) can be used to determine the methylation state of methylation CpG sites.

When performing a bisulfite conversion method to determine the methylation state of methylation CpG sites, DNA obtained from a blood sample (e.g., leukocyte DNA) can be treated with bisulfite, which converts unmethylated cytosines into uracil. The methylated cytosines remain unchanged during the bisulfite treatment. Once the unmethylated cytosines are into uracil, the methylation profile of the DNA can be determined by performing DNA sequencing (e.g., DNA sequencing of PCR amplified products of interest).

Once a human is determined to having altered levels of methylation of methylation CpG sites that are indicative of lung cancer (e.g., small cell lung cancer), then the human can be classified as having lung cancer (e.g., small cell lung cancer) or can be evaluated further to confirm a diagnosis of lung cancer (e.g., small cell lung cancer). Humans identified as having lung cancer (e.g., small cell lung cancer) as described herein can be treated with an appropriate lung cancer (e.g., small cell lung cancer) treatment including, without limitation, surgery, radiation (e.g., stereotactic body radiotherapy), or chemotherapy (e.g., etoposide, irinotecan, cisplatin, carboplatin, vincristine sulfate, cyclophosphamide, doxorubicin, ifosfamide, methotrexate, lomustine, or combinations thereof such as a combination of cyclophosphamide, doxorubicin, and vincristine sulfate or a combination of etoposide with either cisplatin or carboplatin).

This document also provides methods for treating lung cancer patients. For example, a CpG methylation site profile described herein as being indicative of the presence of lung cancer can be detected in a blood sample obtained from a human using the methods and materials provided herein. Such a detection can occur at an early time point in the development of the patient's lung cancer. Once that human is confirmed to have lung cancer, the human can be instructed to undergo lung cancer surgery, radiation treatment (e.g., stereotactic body radiotherapy), chemotherapy effective against lung cancer, or a combination thereof. For example, a human identified as having very early stage lung cancer (e.g., very early stage small cell lung cancer) as described herein can undergo surgery to remove lung cancer tissue followed by a combination of chemotherapy and chest radiation therapy. In some cases, the human can be instructed to undergo brain radiation treatment. Examples of chemotherapy options for treating lung cancer include, without limitation, etoposide, irinotecan, cisplatin, carboplatin, vincristine sulfate, cyclophosphamide, doxorubicin, ifosfamide, methotrexate, lomustine, and combinations thereof such as a combination of cyclophosphamide, doxorubicin, and vincristine sulfate or a combination of etoposide with either cisplatin or carboplatin.

The invention will be further described in the following examples, which do not limit the scope of the invention described in the claims.

### EXAMPLES

### Example 1

### Methylation Markers for Small Cell Lung Cancer in Peripheral Blood Leukocyte DNA

### Sample Recruitment

The methods of identifying and enrolling small-cell lung cancer (SCLC) patients and controls were performed as described elsewhere (Yang et al., Archives of Internal Medicine, 168:1097-1103 (2008); Yang et al., Chest, 128:452-462 (2005); and Yang et al., Cancer Epidemiol Biomarkers Prev., 8:461-465 (1999)). In brief, newly diagnosed cases of lung cancer were identified by a daily electronic pathology reporting system. Once identified, patients that consented were enrolled, their medical records abstracted, and interviews conducted. Overall participation and blood sample donation rates were 87% and 73%, respectively. For controls, community residents identified as having had a general medical examination and as having a leftover blood sample from routine clinical tests, excluding individuals diagnosed with major organ failure (e.g., heart, brain, lung, kidney, or liver) on or prior to their visit, were selected. SCLC cases were identified from among all lung cancer cases. Controls were selected such that the distributions of age, sex, and smoking history were comparable between the cases and controls. Ninety-five percent of the study subjects were white, representing a U.S. mid-western population in and surrounding Minnesota.

### DNA Modification by Sodium Bisulfite

DNA was extracted from 5 mL of whole blood utilizing an automated platform. The whole blood DNA was predominantly derived from leukocytes. Freely circulating DNA in plasma was estimated to account for <0.07 percent of whole blood DNA based on QIAGEN user manual. Thus, the circulating DNA in whole blood was negligible when compared to DNA from leukocytes. The whole blood DNA was referred to as leukocyte DNA throughout this study. The genomic DNA specimens were modified using an EZ DNA Methylation kit from Zymo Research Corporation (Orange, Calif.) that combined bisulfite conversion and DNA clean up. The modification kit was based on the three-step reaction that takes place between cytosine and sodium bisulfite where cytosine is converted into uracil. 1 μg of genomic DNA from peripheral blood DNA was used for the modification under recommendations from the manufacturer. Treated DNA specimens were stored at −20° C. and were assayed within two weeks.

### Methylation Profiling Analysis

The modified DNA specimens were labeled and hybridized with equal numbers of samples from each group, balanced across the entire Beadchip, to avoid confounding study results with processing variance. The arrays were imaged using a BeadArray Reader scanner, which represented each methylation data point as fluorescent signals from the M (methylated) and U (unmethylated) alleles. The proportion methylated (β-value) at each CpG site was calculated using BeadStudio Software (Illumina) after subtracting background intensity, computed from negative controls, from each analytical data point.

### Pyrosequencing Methylation Assays

Primers were designed using Pyrosequencing Assay Design Software (Biotage AB, Uppsala, Sweden). Sequences of the primers are listed in Table 2. The PCR was carried out on 10 ng of bisulfite treated DNA using TaqGold DNA polymerase (Applied Biosystems) under the following conditions: 10 minutes at 95° C., followed by 50 cycles of 35 seconds at 95° C., 35 seconds at 57.5° C., and 1 minute at 72° C. Pyrosequencing reactions were performed on Biotage PyroMark MD System (Biotage AB, Uppsala, Sweden) according to the manufacturer's protocols by the sequential addition of single nucleotides in a predefined order. Raw data were analyzed using Pyro Q-CpG 1.0.9 analysis software (Biotage AB, Uppsala, Sweden).

TABLE 2
Primers for pyrosequencing methylation assay.

| | Primer | | Primer Sequences |
|---|---|---|---|
| Genes | Names | Notes | (from 5′ to 3′) |
| PECAM1 | PECAM1f | PCR-forward, biotin- | TTGAGAAATTAGTTTTGTGAAAAG |
| | | Labeled | |
| | PECAM1r | PCR-reverse | TCAAACCAACCCAAACCCCATTATT |
| | PECAM1sr | sequencing-reverse | TTCCAACCATAACTACCATTACCT |
| S100A2 | S100A2f | PCR-forward | GTTAGTTTTATTATTAGTTGGGGGAGGGT |
| | S100A2r | PCR-reverse, biotin- | ACCCCCATCCAAAATACCC |
| | | Labeled | |
| | S100A2sf | sequencing-forward | GGAAGTGGGAGGTGT |
| ERCC1 | ERCC1f | PCR-forward | GAGTTAGTGTTGGTGATATAGTAGTGA |
| | ERCC1r | PCR-reverse, biotin- | CATCCCAAACCTACCCATTCT |
| | | Labeled | |
| | ERCC1sf | sequencing-forward | TTAAGGTTTAGTAAGGGATATAGATA |
| SLC22A18 | SLC22A18f | PCR-forward | GTGTTTATTTTTAAGATTGGTTGAGGTATT |
| | SLC22A18r | PCR-reverse, biotin- | TCCCCAACCCCAAAACATT |
| | | Labeled | |
| | SLC22A18sf | sequencing-forward | TTAGTTAGTTTGGATTTTTTTAT |
| CSF3R | CSF3Rf | PCR-forward, biotin- | GGGTGTGTTTTAGGTTTTAGGGAATT |
| | | Labeled | |
| | CSF3Rr | PCR-reverse | CCCAAAATTCCTATTTCTCCATCTA |
| | CSF3Rsr | sequencing-reverse | CCTAATCTATAAACAATACACAAA |
| MMP9 | MMP9f2 | PCR-forward | GTTTGGGGTTTTGTTTGATTTG |
| | MMP9r2 | PCR-reverse, biotin- | CCACCCCTCCTTAACAAACAAATAC |
| | | Labeled | |
| | MMP9seqf2 | sequencing-forward | TGATTTGGTAGTGGAGAT |
| EMR3 | EMR3f2 | PCR- forward | ATTTTAGGTTAGTTGATTTATGAAAT |
| | EMR3r2 | PCR-reverse, biotin- | AAATTTACCAACTCAATCATCCCAAAA |
| | | Labeled | |
| | EMR3seqf2 | sequencing-forward | GAAAAGTAAATTGTTTTTTTTTTTT |
| IL-10 | IL-10-f1 | PCR-forward | TGTAAGTTTAGGGAGGTTTTTTTATTTATT |
| | IL-10-r1 | PCR-reverse, biotin- | AATTCATATTCAACCAATCATTTTTACTT |
| | | Labeled | |
| | IL-10-seqf1 | sequencing-forward | AAGTTATAATTAAGGTTTTT |
| CAV1 | CAV1f3 | PCR-forward | AAGGGAAGGTTTAGGATAGGGTAGGATT |
| | CAV1r3 | PCR-reverse, biotin- | TTTTCCCAATACATCATCTCAACA |
| | | Labeled | |
| | CAV1seqfs3 | sequencing-forward | AGGGTAGGATTGTGGAT |
| TRIP6 | TRIP6f2 | PCR-forward | GGGTAGGGGTTGGGGAATT |
| | TRIP6r2 | PCR-reverse, biotin- | ATACCCCCCCCCTACTAAACCC |
| | | Labeled | |
| | TRIP6s2 | sequencing-forward | GAAGGGGATTTTGTGA |

### Data Analysis

Demographic characteristics between cases and controls were summarized and compared using chi-square tests for nominal variables or rank sum tests for the quantitative variables. The percent methylated measurements were summarized by their mean and standard deviation within the two study groups, and analysis of covariance approaches were used to compare the degree of methylation between study groups for each CpG site while adjusting for pack years of smoking. Because of the non-normality of the methylation values, rank-based analyses, which are analogous to rank-sum tests when there are no covariates, were used. After obtaining the p-values for each of the CpG sites in the testing set, false discovery rate (FDR) approaches were employed, and a q-value was computed for each p-value (Storey and Tibshirani, Proc. Natl. Acad. Sci. USA, 100:9440-9445 (2003)). CpG sites with q-values of less than 0.05 were considered to be significant.

In the validation phase, the methylation levels of the 10 selected CpGs were compared between cases and controls in the validation set using the rank-based procedures outlined above. Logistic regression approaches were used to simultaneously assess the association between all nine validation CpGs and case-control status. This multivariable model was further refined via stepwise model selection with the p-value to enter and remain in the model set at 0.05, to determine a CpG set that simultaneously contributes to the discrimination between SCLC cases and controls. As part of these logistic regression analyses, the degree of concordance between model predictions and observed case-control status was measured by extracting estimates of the area under the receiver operating characteristic (ROC) curve. This quantity, often referred to as the c-statistic, examines all possible case control pairs and measures the proportion of the time the statistical model predicts higher risk for the case (Zweig and Campbell, Clin. Chem., 39:561-577 (1993)). All analyses were conducted using the SAS software system (Cary N.C.).

### Results

### Characteristics of Study Subjects

By matching design, no difference in age, sex, and smoking status was found between the cases and controls in both the testing and validation sets. Basic descriptive information of the cases and controls are provided in Table 3. For the testing set, five cases were dropped due to DNA quality issues, and the remaining 39 cases and 44 controls were used in the analysis. There was a greater than 3-year difference between the cases and controls in the mean pack-years of cigarette smoking (60.1 vs. 56.5). However, median pack-years were similar (51 vs. 52), and the test comparing the two groups did not reach statistical significance (p=0.525). To be conservative, the number of pack-years was adjusted in all DNA methylation analyses.

TABLE 3
Characteristics of patients with SCLC and healthy controls.

| | Testing Set | | | | Validation Set | | | |
|---|---|---|---|---|---|---|---|---|
| | Cases | Controls | Total | | Cases | Controls | Total | |
| | (N = 39) | (N = 44) | (N = 83) | p value | (N = 138) | (N = 138) | (N = 276) | p value |
| AGE | | | | 0.5392 | | | | 0.6736 |
| Mean (SD) | 64.8 (6.1) | 65.6 (5.7) | 65.2 (5.8) | | 64.4 (9.54) | 64.8 (9.46) | 64.6 (9.48) | |
| Median | 65.0 | 65.5 | 65.0 | | 66.0 | 66.0 | 66.0 | |
| Q1, Q3 | 61.0, 69.0 | 61.5, 69.0 | 61.0, 69.0 | | 59.0, 71.0 | 59.0, 71.0 | 59.0, 71.0 | |
| Range | (54.0-78.0) | (57.0-78.0) | (54.0-78.0) | | (37.0-85.0) | (33.0-82.0) | (33.0-85.0) | |
| Gender | | | | 0.6470 | | | | 1 |
| Female | 17 (43.6%) | 17 (38.6%) | 34 (41.0%) | | 61 (44.2%) | 61 (44.2%) | 122 (44.2%) | |
| Male | 22 (56.4%) | 27 (61.4%) | 49 (59.0%) | | 77 (55.8%) | 77 (55.8%) | 154 (55.8%) | |
| Cigarette | | | | 0.9072 | | | | 1 |
| Smoking Status | | | | | | | | |
| Never | 0 | 0 | 0 | | 8 (5.8%) | 8 (5.8%) | 16 (5.8%) | |
| Former | 20 (51.3%) | 22 (50.0%) | 42 (50.6%) | | 63 (45.7%) | 63 (45.7%) | 126 (45.7%) | |
| Current | 19 (48.7%) | 22 (50.0%) | 41 (49.4%) | | 67 (48.6%) | 67 (48.6%) | 134 (48.6%) | |
| Pack-Years | | | | 0.5249 | | | | 0.9218 |
| Mean (SD) | 60.1 (25.1) | 56.5 (25.8) | 58.2 (25.4) | | 56.8 (29.4) | 56.4 (29.2) | 56.6 (29.3) | |
| Median | 51.0 | 52.0 | 52.0 | | 52.0 | 52.0 | 52.0 | |
| Q1, Q3 | 42.0, 74.0 | 39.0, 68.3 | 41.0, 72.0 | | 37.0, 75.0 | 36.0, 77.0 | 36.0, 76.5 | |
| Range | (22.0-126.0) | (17.0-141.0) | (17.0-141.0) | | (3.0-146.0) | (3.0-147.0) | (3.0-147.0) | |

### Differentially Methylated CpG Sites

Since the majority of the SCLC patients received radiation treatment or chemotherapy before blood was drawn, the correlations between the time on treatment (as a proxy for treatment intensity) and the degree of methylation was examined to determine if the CpG methylation levels might be affected by treatment in the 39 SCLC patients. Among the 1,505 CpG sites, the length of time on treatment was significantly correlated with the methylation levels of 173 CpGs (p<0.05). While some of these associations may be false positives, but to be conservative, all 173 CpGs were excluded from the analyses. Among the remaining 1332 CpG sites, 922 were located within CpG islands and 410 were in non-CpG islands. Significant differences were observed between SCLC cases and controls at 62 sites in 52 independent genes (FDR<=0.05). Interestingly, only 25 of the 62 sites were in CpG islands, which was significantly lower than the expected 42.9 sites (62×922/1332) (p<0.001, Chi square test). The odds of a significant CpG not being in a CpG island was greater than three times higher than the odds of being in a CpG island (OR=3.56, 95% CI: 2.11-6.00). Furthermore, only six of the 62 sites exhibited an increased level of methylation in SCLC patients, including two in the ITK gene, two in the RUNX3 gene, and one in each of the CTLA4 and PLG genes. Because some methylation differences were small and difficult to reliably detect, the CpG sites with an absolute mean 0 difference of less than 0.03 were excluded, resulting in 43 significant CpG sites of primary interest in 36 independent genes (Table 4).

TABLE 4
Differential methylations between 39 SCLC cases and 44 healthy controls in testing set.

| | | CpG | Adjusted | | Controls | | Cases | | Case/control |
|---|---|---|---|---|---|---|---|---|---|
| Symbol | Illumina CpG ID^{a} | Island | p-value | q-value | Mean β | SD^{b} | Mean β | SD^{b} | Difference |
| SLC22A18 | SLC22A18_P216_R | no | 0.00002 | 0.00877 | 0.464 | 0.091 | 0.354 | 0.131 | −0.11 |
| PADI4 | PADI4_E24_F | no | 0.00002 | 0.00877 | 0.257 | 0.067 | 0.190 | 0.096 | −0.067 |
| MMP9 | MMP9_P189_F | no | 0.00005 | 0.00877 | 0.377 | 0.076 | 0.298 | 0.100 | −0.079 |
| LTB4R | LTB4R_P163_F | no | 0.00005 | 0.00877 | 0.303 | 0.063 | 0.242 | 0.068 | −0.061 |
| S100A2 | S100A2_E36_R | no | 0.00006 | 0.00877 | 0.353 | 0.070 | 0.282 | 0.073 | −0.071 |
| RUNX3 | RUNX3_P247_F | yes | 0.00006 | 0.00877 | 0.703 | 0.102 | 0.790 | 0.153 | 0.087 |
| MPO | MPO_E302_R | no | 0.00007 | 0.00877 | 0.700 | 0.065 | 0.618 | 0.089 | −0.082 |
| IL10 | IL10_P85_F | no | 0.00007 | 0.00877 | 0.229 | 0.051 | 0.178 | 0.076 | −0.051 |
| RUNX3 | RUNX3_E27_R | no | 0.00008 | 0.00885 | 0.862 | 0.049 | 0.900 | 0.092 | 0.038 |
| PECAM1 | PECAM1_E32_R | yes | 0.00008 | 0.00885 | 0.257 | 0.065 | 0.189 | 0.087 | −0.068 |
| EMR3 | EMR3_E61_F | no | 0.00014 | 0.01292 | 0.22 | 0.049 | 0.166 | 0.085 | −0.054 |
| SPI1 | SPI1_E205_F | yes | 0.00017 | 0.01292 | 0.315 | 0.057 | 0.250 | 0.100 | −0.065 |
| TNFRSF1A | TNFRSF1A_P678_F | no | 0.00019 | 0.01292 | 0.754 | 0.070 | 0.662 | 0.125 | −0.092 |
| LMO2 | LMO2_E148_F | no | 0.00019 | 0.01292 | 0.442 | 0.103 | 0.327 | 0.151 | −0.115 |
| IL10 | IL10_P348_F | no | 0.0002 | 0.01292 | 0.64 | 0.086 | 0.509 | 0.180 | −0.131 |
| ERCC1 | ERCC1_P440_R | yes | 0.0002 | 0.01292 | 0.141 | 0.046 | 0.103 | 0.047 | −0.038 |
| CSF3R | CSF3R_P472_F | no | 0.00041 | 0.02392 | 0.371 | 0.097 | 0.276 | 0.134 | −0.095 |
| JAK3 | JAK3_P1075_R | no | 0.00044 | 0.02392 | 0.683 | 0.077 | 0.617 | 0.087 | −0.066 |
| LCN2 | LCN2_P141_R | no | 0.00048 | 0.02392 | 0.789 | 0.078 | 0.708 | 0.131 | −0.081 |
| CD82 | CD82_P557_R | yes | 0.00048 | 0.02392 | 0.277 | 0.097 | 0.192 | 0.109 | −0.085 |
| PI3 | PI3_P274_R | no | 0.00052 | 0.02457 | 0.835 | 0.053 | 0.763 | 0.110 | −0.072 |
| TRIP6 | TRIP6_P1090_F | yes | 0.00053 | 0.02457 | 0.359 | 0.107 | 0.259 | 0.139 | −0.100 |
| TIE1 | TIE1_E66_R | no | 0.00055 | 0.02457 | 0.224 | 0.067 | 0.164 | 0.085 | −0.06 |
| TRIP6 | TRIP6_P1274_R | yes | 0.00061 | 0.02543 | 0.617 | 0.101 | 0.488 | 0.178 | −0.129 |
| CD9 | CD9_P585_R | yes | 0.00063 | 0.02548 | 0.385 | 0.061 | 0.314 | 0.103 | −0.071 |
| SEPT9. | SEPT9_P374_F | yes | 0.00065 | 0.02555 | 0.252 | 0.097 | 0.180 | 0.102 | −0.072 |
| MPL | MPL_P62_F | no | 0.00091 | 0.0319 | 0.492 | 0.098 | 0.389 | 0.151 | −0.103 |
| CASP10 | CASP10_P334_F | no | 0.00094 | 0.0319 | 0.243 | 0.059 | 0.191 | 0.078 | −0.052 |
| AIM2 | AIM2_P624_F | no | 0.00094 | 0.0319 | 0.467 | 0.137 | 0.353 | 0.178 | −0.114 |
| SEPT9. | SEPT9_P58_R | yes | 0.001 | 0.03234 | 0.929 | 0.046 | 0.888 | 0.061 | −0.041 |
| CSF1R | CSF1R_E26_F | no | 0.00107 | 0.03239 | 0.749 | 0.075 | 0.662 | 0.135 | −0.087 |
| CSF3R | CSF3R_P8_F | no | 0.0013 | 0.03635 | 0.225 | 0.072 | 0.168 | 0.096 | −0.057 |
| MMP14 | MMP14_P13_F | yes | 0.00167 | 0.04137 | 0.500 | 0.101 | 0.400 | 0.157 | −0.100 |
| BTK | BTK_P105_F | no | 0.00167 | 0.04137 | 0.132 | 0.047 | 0.100 | 0.050 | −0.032 |
| GRB7 | GRB7_E71_R | no | 0.00178 | 0.04319 | 0.374 | 0.092 | 0.296 | 0.134 | −0.078 |
| STAT5A | STAT5A_P704_R | no | 0.00189 | 0.04428 | 0.183 | 0.06 | 0.139 | 0.059 | −0.044 |
| NOTCH4 | NOTCH4_E4_F | no | 0.00189 | 0.04428 | 0.170 | 0.069 | 0.123 | 0.072 | −0.047 |
| HOXB2 | HOXB2_P99_F | yes | 0.00214 | 0.04667 | 0.552 | 0.09 | 0.483 | 0.103 | −0.069 |
| MFAP4 | MFAP4_P197_F | no | 0.0022 | 0.04721 | 0.244 | 0.061 | 0.196 | 0.080 | −0.048 |
| SLC5A5 | SLC5A5_E60_F | yes | 0.00227 | 0.04721 | 0.527 | 0.089 | 0.459 | 0.107 | −0.068 |
| CD34 | CD34_P339_R | no | 0.00227 | 0.04721 | 0.268 | 0.049 | 0.236 | 0.056 | −0.032 |
| MFAP4 | MFAP4_P10_R | no | 0.00249 | 0.04933 | 0.172 | 0.061 | 0.133 | 0.059 | −0.039 |
| EMR3 | EMR3_P39_R | no | 0.00264 | 0.04933 | 0.287 | 0.064 | 0.232 | 0.072 | −0.055 |
| CAV1 | CAV1_P169_F^{c} | yes | 0.35439 | 0.63028 | 0.191 | 0.056 | 0.176 | 0.054 | −0.015 |

^{a}The CpG IDs in bold are selected to run pyrosequencing for validation.
^{b}SD—standard deviation.
^{c}The CpG site in the gene CAV1 was selected as negative control.

### Validation of Selected CpG Sites by Pyrosequencing Methylation Assay

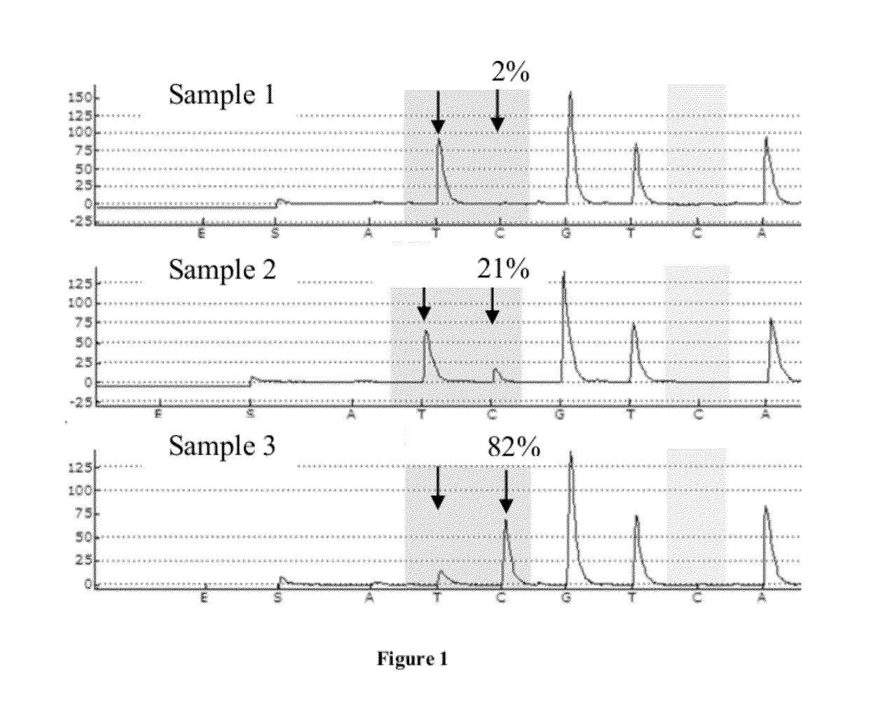

Based on three major parameters (FDR q values, number of significant CpGs/gene, and mean difference between groups), ten CpG sites were selected including nine significant CpGs (FDR<0.05) for validation and one non-significant CpG (FDR>0.05). These CpG sites were located in ten different genes (IL10, PECAM1, S100A2, MMP9, ERCC1, EMR3, SLC22A18, TRIPE, CSF3R, and CAV1), with CAV1 serving as a negative control. A new assay was designed for each of the ten CpG sites using pyrosequencing technology as described elsewhere (Tost and Gut, Methods Mol. Biol., 373:89-102 (2007); and Tost and Gut, Nat. Protoc., 2:2265-2275 (2007)). FIG. 1 shows methylation levels of a CpG site, 85 bp upstream to the transcription start site in the gene, IL10, in three different samples.

The ten CpG sites were then tested for methylation differences, again in peripheral blood DNA specimens from a validation set between 138 SCLC cases and 138 matched controls (Table 3, right panel). The nine testing-set-positive CpG sites again demonstrated significant differences (all p-values <0.0003, Table 5), while the negative control CpG site only differed between the validation set of the cases and controls in an absolute percent methylated by less than 1 percent. This small difference did not reach statistical significance.

TABLE 5
Differential methylations between 138 SCLC cases and 138 matched controls for validation study.

| | | | | Control | | Case | | |
|---|---|---|---|---|---|---|---|---|
| | | | | Mean | | Mean | | |
| Gene | | CpG | Adjusted | methylation | | methylation | | Case/control |
| Symbol | Illumina CpG IDs | Island | p-value | level | SD^{a} | level | SD^{a} | Difference |
| IL10 | IL10_P85_F | no | <0.0001 | 0.116 | 0.032 | 0.077 | 0.035 | −0.039 |
| PECAM1 | PECAM1_E32_R | yes | <0.0001 | 0.343 | 0.089 | 0.242 | 0.104 | −0.101 |
| S100A2 | S100A2_E36_R | no | <0.0001 | 0.288 | 0.076 | 0.211 | 0.063 | −0.077 |
| MMP9 | MMP9_P189_F | no | <0.0001 | 0.058 | 0.020 | 0.037 | 0.021 | −0.021 |
| ERCC1 | ERCC1_P440_R | yes | <0.0001 | 0.135 | 0.044 | 0.085 | 0.037 | −0.050 |
| EMR3 | EMR3_E61_F | no | <0.0001 | 0.161 | 0.043 | 0.115 | 0.050 | −0.046 |
| SLC22A18 | SLC22A18_P216_R | no | <0.0001 | 0.227 | 0.059 | 0.155 | 0.074 | −0.072 |
| TRIP6 | TRIP6_P1090_F | yes | 0.0003 | 0.44 | 0.255 | 0.319 | 0.259 | −0.121 |
| CSF3R | CSF3R_P472_F | no | <0.0001 | 0.277 | 0.066 | 0.18 | 0.084 | −0.097 |
| CAV1 | CAV1_P169_F | yes | 0.3577 | 0.1 | 0.063 | 0.109 | 0.07 | 0.009 |

^{a}SD-standard deviation.

### CpG Methylation Patterns and Risk Prediction of SCLC Using Logistic Regression Models

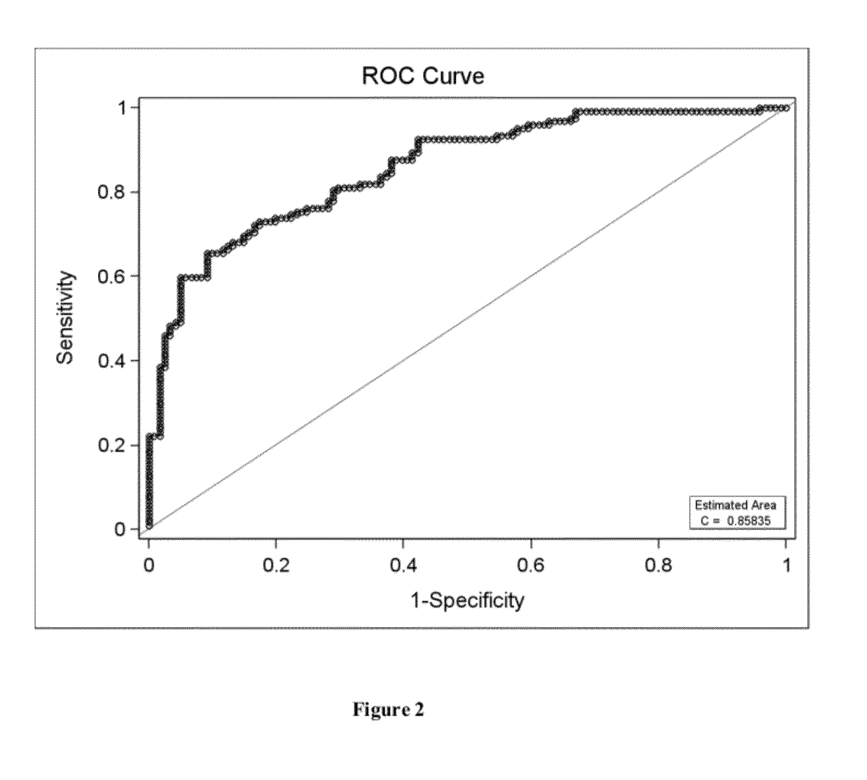

Based on the nine validated CpG sites accounting for age, sex, and smoking history, the model provided herein had an area under the ROC curve of 0.858 (FIG. 2), suggesting the model correctly classified SCLC cases as being at a higher risk than controls for 85.8% of case-control pairs. Further stepwise selection identified two of the nine sites, one in CSF3R and the other in ERCC1, contributing independent information to discriminate cases from controls. Specifically, for each five-percent decrease in the methylation level of ERCC1, there was an approximately four-fold (OR=3.9, 95% CI: 2.0-6.1, p<0.001) increase in the odds ratio of SCLC. For each five-percent methylation decrease of CSF3R, there was a 1.5-fold higher odds ratio of SCLC (OR=1.5, 95% CI: 1.1-2.0, p=0.008).

The results provided herein indicate that methylation status of peripheral blood DNA, a stable and easily accessible material, can be reliably used for risk assessment and diagnosis of SCLC. In addition, the results provided herein demonstrate that methylation levels in the tested CpG of an imprinting gene, SLC22A18, have a strong association with SCLC in both the testing and validation sets (adjusted p<0.0001, Tables 3 and 5). It is noted that only one to two CpG sites per gene that are predefined by the manufacturer for inclusion on the methylation array used (Illumina Inc.) were tested. The tested CpGs are not necessarily most representative for a particular gene. Additional analysis can confirm that the ability to use other CpGs as described herein for these genes. Nevertheless, the results provided herein demonstrate that methylation differences between SCLC patients and controls are present and can be reliably detected in peripheral blood leukocyte DNA. The successful use of the easily accessible specimen (e.g., DNA from peripheral blood leukocytes) in this study can significantly expand the research application from genetics (such as genome-wide association studies) to epigenetics (such as epigenome-wide association studies). For example, the methylation panels provided herein can be used as second-tier disease prediction or non-invasive detection tools among high-risk individuals, particularly smokers with equivocal findings from CT screening.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for identifying a human as having small cell lung cancer, wherein said method comprises:
(a) performing a bisulfite conversion using nucleic acid obtained from a blood sample of a human to detect at least three methylation CpG sites that have an altered methylation status indicative of small cell lung cancer, wherein said at least three methylation CpG sites are selected from the group consisting of the CpG methylation sites listed in Table 1, and
(b) classifying said human as having small cell lung cancer based at least in part on said detection of said at least three methylation CpG sites that have an altered methylation status indicative of small cell lung cancer.

2. The method of claim 1, wherein said blood sample is a blood sample obtained from a human not subjected to a prior lung tissue biopsy.

3. The method of claim 1, wherein said method comprises performing said bisulfite conversion using said nucleic acid to detect at least four methylation CpG sites selected from said group that have an altered methylation status indicative of small cell lung cancer.

4. The method of claim 3, wherein said at least four methylation CpG sites are selected from the group consisting of IL10_P85_F, PECAM1_E32_R, S100A2_E36_R, MMP9_P189_F, ERCC1_P440_R, EMR3_E61_F, SLC22A18_P216_R, TRIP6_P1090_F, and CSF3R_P472_F CpG methylation sites.

5. The method of claim 1, wherein said method comprises performing said bisulfite conversion using said nucleic acid to detect at least five methylation CpG sites selected from said group that have an altered methylation status indicative of small cell lung cancer.

6. The method of claim 5, wherein said at least five methylation CpG sites are selected from the group consisting of IL10_P85_F, PECAM1_E32_R, S100A2_E36_R, MMP9_P189_F, ERCC1_P440_R, EMR3_E61_F, SLC22A18_P216_R, TRIP6_P1090_F, and CSF3R_P472_F CpG methylation sites.

7. The method of claim 1, wherein said method comprises performing said bisulfite conversion using said nucleic acid to detect IL10_P85_F, PECAM1_E32_R, S100A2_E36_R, MMP9_P189_F, ERCC1_P440_R, EMR3_E61_F, SLC22A18P216_R, TRIP6_P1090_F, and CSF3R_P472_F CpG methylation sites that have an altered methylation status indicative of small cell lung cancer.

8. A method for treating small cell lung cancer, wherein said method comprises:
(a) detecting the presence of at least three methylation CpG sites that have an altered methylation status indicative of small cell lung cancer in nucleic acid obtained from a blood sample of a human, wherein said at least three methylation CpG sites are selected from the group consisting of the CpG methylation sites listed in Table 1, and
(b) administering a cancer radiation treatment, a cancer chemotherapeutic agent, or a combination thereof to said human.

9. The method of claim 8, wherein said blood sample is a blood sample obtained from a human not subjected to a prior lung tissue biopsy.

10. The method of claim 8, wherein said method comprises detecting the presence of at least four methylation CpG sites selected form said group that have an altered methylation status indicative of small cell lung cancer in said nucleic acid.

11. The method of claim 10, wherein said at least four methylation CpG sites are selected from the group consisting of IL10_P85_F, PECAM1_E32_R, S100A2_E36_R, MMP9_P189_F, ERCC1_P440_R, EMR3_E61_F, SLC22A18_P216_R, TRIP6_P1090_F, and CSF3R_P472_F CpG methylation sites.

12. The method of claim 8, wherein said method comprises detecting the presence of at least five methylation CpG sites selected from said group that have an altered methylation status indicative of small cell lung cancer in said nucleic acid.

13. The method of claim 12, wherein said at least five methylation CpG sites are selected from the group consisting of IL10_P85_F, PECAM1_E32_R, S100A2_E36_R, MMP9_P189_F, ERCC1_P440_R, EMR3_E61_F, SLC22A18_P216_R, TRIP6_P1090_F, and CSF3R_P472_F CpG methylation sites.

14. The method of claim 8, wherein said method comprises detecting the presence of IL10_P85_F, PECAM1_E32_R, S100A2_E36_R, MMP9_P189_F, ERCC1_P440_R, EMR3_E61_F, SLC22A18_P216_R, TRIP6_P1090_F, and CSF3R_P472_F CpG methylation sites that have an altered methylation status indicative of small cell lung cancer in said nucleic acid.

15. The method of claim 8, wherein said method comprises administering said cancer radiation treatment.

16. The method of claim 15, wherein said cancer radiation treatment comprises stereotactic body radiotherapy.

17. The method of claim 8, wherein said method comprises administering said cancer chemotherapeutic agent.

18. The method of claim 17, wherein said cancer chemotherapeutic agent is etoposide, irinotecan, cisplatin, carboplatin, vincristine sulfate, or a combination thereof.


SEQUENCE LISTING (73 sequences)

SEQ ID NO: 1 - DNA, 53 bp, Homo sapiens
gtcagcctggatcctctcatccggcagaactgtcgccttgcttctctgaagcg

SEQ ID NO: 2 - DNA, 53 bp, Homo sapiens
tcctacagccagagggacgagctagcccgacgatggcccaggggacattgatc

SEQ ID NO: 3 - DNA, 45 bp, Homo sapiens
ttgcctgacttggcagtggagactgcgggcagtggagagaggagg

SEQ ID NO: 4 - DNA, 47 bp, Homo sapiens
ggggaagaaaggccatcaaggtagatgcgggtggggaacagcttgag

SEQ ID NO: 5 - DNA, 42 bp, Homo sapiens
cacagtgggaagtgggaggtgtcgtggggactgggcatcctg

SEQ ID NO: 6 - DNA, 53 bp, Homo sapiens
cggccttggctcattggctgggccgcggtcacctgggccgtgatgtcacggcc

SEQ ID NO: 7 - DNA, 50 bp, Homo sapiens
ggagcagcaccttcagagggctggggcgtggccagaatggccaggagccc

SEQ ID NO: 8 - DNA, 50 bp, Homo sapiens
agccacaatcaaggtttcccggcacaggattttttctgcttagagctcct

SEQ ID NO: 9 - DNA, 48 bp, Homo sapiens
cggcagccagggtggaggagctccgaagctgacagagcagagtgggcc

SEQ ID NO: 10 - DNA, 51 bp, Homo sapiens
gcgcctgcagagagaccggctgtggcgctggtcaggtaatggcagccatgg

SEQ ID NO: 11 - DNA, 53 bp, Homo sapiens
agcaaactgcttcccctctttcgccatcagactcatggttctgcttttcgttt

SEQ ID NO: 12 - DNA, 48 bp, Homo sapiens
gggaaacccttccattttgcacgcctgtaacatccagccgggctccga

SEQ ID NO: 13 - DNA, 50 bp, Homo sapiens
tcctggctctgccaccaatcatgcgacatcaggcaactcctctcctaagc

SEQ ID NO: 14 - DNA, 51 bp, Homo sapiens
cggagccttcacccttgcagcgagctctctcacaccagatgtgctctgcgt

SEQ ID NO: 15 - DNA, 54 bp, Homo sapiens
attcgcgtgttcctaggtcacagtgacgtggacaaattgcccattccagaatac

SEQ ID NO: 16 - DNA, 56 bp, Homo sapiens
gagcttacggttcagtaagggacacagacacgttcccagtgctgacccagaatggg

SEQ ID NO: 17 - DNA, 53 bp, Homo sapiens
ctcactgctcccctcttcattacgtattctgtgcattgcccatagaccaggca

SEQ ID NO: 18 - DNA, 49 bp, Homo sapiens
ggacaggcacagactggaacttggacccgaggcaggacagggagctggc

SEQ ID NO: 19 - DNA, 45 bp, Homo sapiens
aatgtccctcactctccccgtccctctgtcttgcccaatcctgac

SEQ ID NO: 20 - DNA, 47 bp, Homo sapiens
aaagttcctgggcccaggccgcctcctgatagaggccccgacttagg

SEQ ID NO: 21 - DNA, 58 bp, Homo sapiens
tctaccagtgacttgctgaataaccttcggtgattcctttctcttcttgggtctcact

SEQ ID NO: 22 - DNA, 45 bp, Homo sapiens
aaggggactttgtgaacagtgggcggggagacgcagaggcagagg

SEQ ID NO: 23 - DNA, 57 bp, Homo sapiens
ccagctcgtcctggctggcctgggtcggcctctggagtatggtctggcgggtgcccc

SEQ ID NO: 24 - DNA, 54 bp, Homo sapiens
cttgggcatggtgcccgcttggcatagcgcccggctccggatcttcctgtgcct

SEQ ID NO: 25 - DNA, 46 bp, Homo sapiens
ctgtcatcccacccagactgcgcgcttctaattcctcctaccccac

SEQ ID NO: 26 - DNA, 48 bp, Homo sapiens
ggggccagcccaggacagaggaaggcgaggcaggcacgcaggaactgg

SEQ ID NO: 27 - DNA, 44 bp, Homo sapiens
aggggcagggacagggacaggacgtggggctgtatctgacagga

SEQ ID NO: 28 - DNA, 57 bp, Homo sapiens
tgtggacataagaaagggttaacatggccgacaactatttcatgagctttttggctt

SEQ ID NO: 29 - DNA, 51 bp, Homo sapiens
gtcagcagtcagccaagttttcgaccatcttggctttaaccagttgcggcc

SEQ ID NO: 30 - DNA, 45 bp, Homo sapiens
ccggtggtctgccggactcctcggggcccacttcgggccctctct

SEQ ID NO: 31 - DNA, 52 bp, Homo sapiens
ttctcctcacttcgtgctctcacgcttttggacactctgtctgcccttctcc

SEQ ID NO: 32 - DNA, 50 bp, Homo sapiens
gcttctctccccgagctctgtcgttaatggctcagcctctgacaggcccg

SEQ ID NO: 33 - DNA, 49 bp, Homo sapiens
agggagggaccagaggagagagcgagagagggaaccagaccccagttcg

SEQ ID NO: 34 - DNA, 50 bp, Homo sapiens
gcagcatgctatctggttccctgctgccgtccctattccaccccctcaac

SEQ ID NO: 35 - DNA, 51 bp, Homo sapiens
gcctctgacttctctgtccgaagtcgggacaccctcctaccacctgtagag

SEQ ID NO: 36 - DNA, 49 bp, Homo sapiens
cagccaccgacaggctgcatgacggtggcaaagtcacttcccctctctg

SEQ ID NO: 37 - DNA, 54 bp, Homo sapiens
cctcggcctgctgcaagcctcacgtctgagctgtttcctgagtcacacaatgtc

SEQ ID NO: 38 - DNA, 54 bp, Homo sapiens
tctattaaacccaggactccagcgaaattacagggaattcgtggtcacgggacc

SEQ ID NO: 39 - DNA, 58 bp, Homo sapiens
gaccacctgtgtctcattagtcctgtcgggcaaagtactgcagacgttaactccctgc

SEQ ID NO: 40 - DNA, 48 bp, Homo sapiens
ggacagacagccggctgcatgggacagcggaacccagagtgagagggg

SEQ ID NO: 41 - DNA, 52 bp, Homo sapiens
atcctgtgctgtgtgtgagtgaagcgtcaggagtgagcaggtatacgtgact

SEQ ID NO: 42 - DNA, 51 bp, Homo sapiens
tgctcagagtggctgggtgtctgcggccccagactgcaaccgcccagagtt

SEQ ID NO: 43 - DNA, 54 bp, Homo sapiens
gggatgattgagttggtaaaccctaacgaggaaatgccctgaaagttacatcac

SEQ ID NO: 44 - DNA, 24 bp, Artificial Sequence
  Description of Artificial Sequence Synthetic primer
ttgagaaattagttttgtgaaaag

SEQ ID NO: 45 - DNA, 25 bp, Artificial Sequence
  Description of Artificial Sequence Synthetic primer
tcaaaccaacccaaaccccattatt

SEQ ID NO: 46 - DNA, 24 bp, Artificial Sequence
  Description of Artificial Sequence Synthetic primer
ttccaaccataactaccattacct

SEQ ID NO: 47 - DNA, 29 bp, Artificial Sequence
  Description of Artificial Sequence Synthetic primer
gttagttttattattagttgggggagggt

SEQ ID NO: 48 - DNA, 19 bp, Artificial Sequence
  Description of Artificial Sequence Synthetic primer
acccccatccaaaataccc

SEQ ID NO: 49 - DNA, 15 bp, Artificial Sequence
  Description of Artificial Sequence Synthetic primer
ggaagtgggaggtgt

SEQ ID NO: 50 - DNA, 27 bp, Artificial Sequence
  Description of Artificial Sequence Synthetic primer
gagttagtgttggtgatatagtagtga

SEQ ID NO: 51 - DNA, 21 bp, Artificial Sequence
  Description of Artificial Sequence Synthetic primer
catcccaaacctacccattct

SEQ ID NO: 52 - DNA, 26 bp, Artificial Sequence
  Description of Artificial Sequence Synthetic primer
ttaaggtttagtaagggatatagata

SEQ ID NO: 53 - DNA, 30 bp, Artificial Sequence
  Description of Artificial Sequence Synthetic primer
gtgtttatttttaagattggttgaggtatt

SEQ ID NO: 54 - DNA, 19 bp, Artificial Sequence
  Description of Artificial Sequence Synthetic primer
tccccaaccccaaaacatt

SEQ ID NO: 55 - DNA, 23 bp, Artificial Sequence
  Description of Artificial Sequence Synthetic primer
ttagttagtttggatttttttat

SEQ ID NO: 56 - DNA, 26 bp, Artificial Sequence
  Description of Artificial Sequence Synthetic primer
gggtgtgttttaggttttagggaatt

SEQ ID NO: 57 - DNA, 25 bp, Artificial Sequence
  Description of Artificial Sequence Synthetic primer
cccaaaattcctatttctccatcta

SEQ ID NO: 58 - DNA, 24 bp, Artificial Sequence
  Description of Artificial Sequence Synthetic primer
cctaatctataaacaatacacaaa

SEQ ID NO: 59 - DNA, 22 bp, Artificial Sequence
  Description of Artificial Sequence Synthetic primer
gtttggggttttgtttgatttg

SEQ ID NO: 60 - DNA, 25 bp, Artificial Sequence
  Description of Artificial Sequence Synthetic primer
ccacccctccttaacaaacaaatac

SEQ ID NO: 61 - DNA, 18 bp, Artificial Sequence
  Description of Artificial Sequence Synthetic primer
tgatttggtagtggagat

SEQ ID NO: 62 - DNA, 26 bp, Artificial Sequence
  Description of Artificial Sequence Synthetic primer
attttaggttagttgatttatgaaat

SEQ ID NO: 63 - DNA, 27 bp, Artificial Sequence
  Description of Artificial Sequence Synthetic primer
aaatttaccaactcaatcatcccaaaa

SEQ ID NO: 64 - DNA, 25 bp, Artificial Sequence
  Description of Artificial Sequence Synthetic primer
gaaaagtaaattgtttttttttttt

SEQ ID NO: 65 - DNA, 30 bp, Artificial Sequence
  Description of Artificial Sequence Synthetic primer
tgtaagtttagggaggtttttttatttatt

SEQ ID NO: 66 - DNA, 29 bp, Artificial Sequence
  Description of Artificial Sequence Synthetic primer
aattcatattcaaccaatcatttttactt

SEQ ID NO: 67 - DNA, 20 bp, Artificial Sequence
  Description of Artificial Sequence Synthetic primer
aagttataattaaggttttt

SEQ ID NO: 68 - DNA, 28 bp, Artificial Sequence
  Description of Artificial Sequence Synthetic primer
aagggaaggtttaggatagggtaggatt

SEQ ID NO: 69 - DNA, 24 bp, Artificial Sequence
  Description of Artificial Sequence Synthetic primer
ttttcccaatacatcatctcaaca

SEQ ID NO: 70 - DNA, 17 bp, Artificial Sequence
  Description of Artificial Sequence Synthetic primer
agggtaggattgtggat

SEQ ID NO: 71 - DNA, 19 bp, Artificial Sequence
  Description of Artificial Sequence Synthetic primer
gggtaggggttggggaatt

SEQ ID NO: 72 - DNA, 22 bp, Artificial Sequence
  Description of Artificial Sequence Synthetic primer
ataccccccccctactaaaccc

SEQ ID NO: 73 - DNA, 16 bp, Artificial Sequence
  Description of Artificial Sequence Synthetic primer
gaaggggattttgtga